(19) United States
(12) Patent Application Publication
(10) Pub. No.: US 2008/0026056 A1
(43) Pub. Date: Jan. 31, 2008

(54) Antibiotic-Based Pharmaceutical Formulation in Microcapsular Form

(75) Inventors: Florence Guimberteau, Montussan (FR); Catherine Castan, Orlienas (FR); Remi Meyrueix, Lyon (FR); Gerard Soula, Meyzieu (FR)

Correspondence Address: PATTON BOGGS LLP, 8484 WESTPARK DRIVE, SUITE 900, MCLEAN, VA, 22102, US

(73) Assignee: Flamel Technologies, Venissieux (FR)

(21) Appl. No.: 11/631,030

(22) PCT Filed: May 25, 2005
(86) PCT No.: PCT/FR05/50366

§ 371 Date: Jun. 28, 2007

(30) Foreign Application Priority Data
Jun. 28, 2004 (FR) 0451353

(51) Int. Cl. A61K 9/50; A61K 31/43; A61P 31/04

(52) U.S. Cl. 424/464; 424/489; 424/490; 514/192

(57) ABSTRACT

The invention relates to oral antibiotic drugs. The object of the invention is to limit or even stop the increase in antibiotic resistance without sacrificing the requirements of (a) increased efficacy of oral antibiotics, particularly for pediatric applications, (b) tolerance, (c) broad spectra of activity, and (d) good patient compliance. This object is achieved by the invention, which proposes the use of modified-release microcapsules, comprising a core that contains at least one active principle AP1 formed of at least one antibiotic, and a coating for said core that governs the modified release of said active principle, for the manufacture of a drinkable or orally dispersible antibiotic pharmaceutical formulation that makes it possible to limit the increase in the antibiotic resistance of the target germs, this formulation being: capable of administration in one or two, preferably two, intakes per day, and definable as follows, relative to an immediate-release oral formulation (IRF*) comprising at least one active principle API, and for the same dose D of API as IRF*:
T_{mic}>T*_{mic}of IRF*

The invention relates to the field of pharmaceutical formulations, especially pediatric or geriatric formulations, for the oral administration of antibiotics (for example amoxicillin optionally associated with clavulanic acid and/or at least one of its salts) for the treatment of bacterial infections, especially those caused by antibiotic-resistant germs such as Streptococcus pneumoniae. The invention further relates to the use of antibiotics (for example amoxicillin optionally associated with clavulanic acid and/or at least one of its salts) for the preparation of “multimicrocapsular” pharmaceutical formulations, especially pediatric or geriatric formulations.

Throughout the present disclosure, the general problem addressed by the invention, which is the improvement of oral antibiotic therapeutic agents in order to prevent or limit the increase in the antibiotic resistance of germs, will be illustrated by the particular case of amoxicillin, although this does not imply a limitation.

Amoxicillin is a known antibiotic belonging to the β-lactam family. Clavulanic acid and its salts, particularly potassium clavulanate, are known β-lactamase inhibitors. It is known that clavulanate improves the efficacy of amoxicillin against resistant microorganisms, including Streptococcus pneumoniae in particular, which is the germ most commonly implicated in respiratory tract and ear infections in pediatrics, sinusitis in patients of all ages, and pneumonia and acute bronchitis in adults.

The combination of amoxicillin and clavulanate exists in the form of aqueous solutions or suspensions (for example a flavored syrup or a powder to be reconstituted into a suspension), but also in the form of tablets.

The improvement in efficacy afforded by this combination can be assessed by resorting to two pharmacokinetic parameters traditionally used for β-lactams, including amoxicillin, namely:
a. the time T_{mic} for which the plasma concentration is greater than or equal to the MIC, the MIC being the Minimum Inhibitory Concentration for a given antibiotic;
b. the maximum plasma concentration (Cmax).

For various β-lactams, a bacteriological cure rate of 85 to 100% is achieved when the serum concentrations exceed the MIC for a period greater than about 40% of the administration interval (CRAIG and ANDES, Ped. Inf. Dis. J., 1996, 15, 255, 259), i.e. T_{mic}≈4.8 hours for an administration interval of 12 hours.

However, one is forced to note that in general terms this cover period does not make it possible to stop the increase in the resistance of germs. The consequence is a regular increase in the minimum inhibitory concentration (MIC) and a regular increase in the prescribed doses. This phenomenon poses a major public health problem because eventually the proposed treatments will become less and less effective.

To maintain the antibiotic concentration at a high value for as long as possible, the practice is to administer the drug at frequent intervals, typically three times a day. However, this type of dosage is generally very restricting and is inapplicable in the case of children. In fact, the regulations in establishments that accommodate children (creches, schools, outdoor centers, etc.) do not allow drugs to be administered during the day.

The outcome is that the drug can only be administered twice a day.

In this context, WO-A-97/09042 proposes the administration of a high dose of amox twice a day in order to increase the T_{mic} and Cmax of the amoxicillin. Said document teaches the use of amoxicillin and clavulanate in a nominal weight ratio of 14:1 to produce a drug for oral administration to pediatric patients, in the form of a powder or a granular product to be reconstituted into a suspension or solution that is suitable for administration twice a day, at high doses of 75 to 115 mg/kg of amoxicillin per day and 5 to 7.5 mg/kg of clavulanate per day, for the treatment of respiratory tract infections.

The strategy of the invention according to WO-A-97/09042 is therefore to increase the dose so as to increase the protection period.

Although this treatment is relatively effective for treating infections, it offers no solution to the problem of stopping the gradual increase in the antibiotic resistance of germs.

Now, the resistance levels (expressed by the MICs) are continuing to increase. For example, 10% of pneumococci currently have an MIC of 2 μg/ml of amoxicillin.

The bacterial resistance is assessed especially by way of the penicillin resistance according to the following criteria, which are those commonly accepted by the scientific community:
the sensitive strains have MICs of ≦0.06 μg/ml,
the intermediate resistance corresponds to an MIC of between 0.12 and 1.0 μg/ml,
and the penicillin resistance is defined by an MIC of ≧2 μg/ml.

The teaching of WO-A-97/09042 confirms what the scientific community also agrees, namely that the problem of the increase in bacterial resistance to antibiotics is all the more serious because the process of increasing the MICs of antibiotics, and hence the doses, is an inexorable one. This leads to a correlative decrease in the therapeutic cover period. Thus, if no action is taken, this could result in a dramatic inefficacy of all known antibiotics in a few years' time.

Furthermore, it is known that infectious germs multiply exponentially and rapidly, e.g. typically in only a few tens of minutes. The mutations which confer antibiotic resistance on the target germs take place within this process of exponential multiplication. The risk of mutation increases with the time for which the growing germs are exposed to the antibiotic in question at a concentration below the MIC. The increase in T_{mic} is therefore crucial not only for treating the patient effectively, but also in particular for stopping the insidious increase in the antibiotic resistance of the target germs.

Moreover, the high dosage according to WO-A-97/09042 is liable to cause a number of unwanted side effects, including especially sneezing, vomiting, contact dermatitis, fever, diarrhea and erythema, among others.

Another technical solution for increasing the T_{mic} is described in WO-A-00/61116, which relates to a multilayer tablet comprising an immediate-release layer of amoxicillin and potassium clavulanate and a slow-release layer of amoxicillin, the tablet being coated with a film of hydroxypropyl methyl cellulose. This tablet comprises e.g. 1000 mg of amoxicillin and 62.5 mg of potassium clavulanate. The recommended dosage is 2×2 tablets per day. This “tablet” form makes it possible to increase the T_{mic} and Cmax of the amoxicillin by varying the sustained release of the amoxicillin and the increase in gastric residence time provided by a large monolithic tablet. There is no way in which these enormous tablets can be administered to children, such a procedure being prohibited by the regulations. Whatever the case may be, it is well known that the taking of 4 very large tablets poses serious administration problems, particularly for the elderly or patients with a physical impediment.

WO-A-03/084517 discloses liquid pharmaceutical formulations that can be administered orally for the modified release of amoxicillin. These formulations consist of suspensions of coated amoxicillin particles (microcapsules), the coating composition of said microcapsules being designed so that the microcapsules have properties for the modified release of amoxicillin that are not perturbed by the aqueous liquid phase of the suspension, said liquid phase also being saturated with amoxicillin. The coating of these microcapsules of amoxicillin comprises e.g. 70% of ethyl cellulose, 23% of polyvinylpyrrolidone and 7% of castor oil. A surfactant/lubricant of the magnesium stearate type may be incorporated in this coating. The invention according to WO-A-03/084517 is concerned with the problem of the stability of microcapsules of amoxicillin in aqueous suspension. In no way does said document consider using said microcapsules for stopping the increase in the antibiotic resistance of germs, which appears to be a major public health problem.

It is therefore evident from this review of the prior art that the technical proposals known hitherto do not provide satisfactory solutions to the dual problem of combating the increase in bacterial resistance to antibiotics and of increasing the efficacy of oral antibiotics, particularly for pediatric applications.

One of the essential objects of the present invention is therefore to provide an oral pharmaceutical formulation, especially pediatric formulation, which is based on at least one active principle selected from antibiotics (for example amoxicillin optionally associated with clavulanic acid and/or at least one of its salts) and which overcomes this deficiency.

Another essential object of the present invention is to provide a modified-release pharmaceutical formulation, especially pediatric formulation, for oral administration, which is based on at least one active principle selected from antibiotics (for example amoxicillin optionally associated with clavulanic acid and/or at least one of its salts) and which makes it possible to maximize the T_{mic} at a given dose of antibiotic active principle, irrespective of the number of intakes.

Another essential object of the present invention is to provide a modified-release pharmaceutical formulation, especially pediatric formulation, for oral administration, which is based on at least one active principle selected from antibiotics (for example amoxicillin optionally associated with clavulanic acid and/or at least one of its salts) and which is not monolithic (tablet).

Another essential object of the present invention is to provide a pediatric modified-release antibiotic formulation which can be administered orally once or twice a day (for example based on amoxicillin optionally associated with clavulanic acid and/or at least one of its salts) and which offers a therapeutic cover that is sufficient to eradicate the germs responsible for the infection, while at the same time limiting or even stopping the increase in the antibiotic resistance of said germs.

Another essential object of the present invention is to provide a modified-release pharmaceutical formulation, especially pediatric formulation, for oral administration, which is based on at least one active principle selected from antibiotics (for example amoxicillin optionally associated with clavulanic acid and/or at least one of its salts) and which makes it possible, for a given type of microcapsule, to release the antibiotic continuously to give a monomodal plasma concentration profile.

Another essential object of the present invention is to provide a modified-release pharmaceutical formulation, especially pediatric formulation, for oral administration, which is based on at least one active principle selected from antibiotics (for example amoxicillin optionally associated with clavulanic acid and/or at least one of its salts) and which is very effective in combating bacterial infections due to resistant germs of the S. pneumoniae type, without a prohibitive increase in the doses such as to cause unwanted side effects.

Another essential object of the present invention is to provide a pharmaceutical formulation of amoxicillin (with or without clavulanate or the like) whose T_{mic} of 4 μg/ml of amoxicillin represents at least 40% of a twelve-hour administration interval, preferably at least 60% and particularly preferably at least 80%, for amoxicillin doses of between 70 and 130 mg/kg/day.

Another essential object of the present invention is to provide an oral pharmaceutical formulation, especially pediatric formulation, for the modified release of amoxicillin and clavulanate which makes it possible to obtain a pharmacokinetic parameter T_{mic} that is greater than or equal to 40% of a twelve-hour administration interval, for an MIC of 4 μg/ml of amoxicillin and for doses below 90 mg/kg/day, preferably of between 50 and 80 mg/kg/day.

Another essential object of the present invention is to propose the use of at least one active principle selected from antibiotics (e.g. amoxicillin optionally associated with clavulanic acid and/or at least one of its salts) for the preparation of a pharmaceutical formulation as defined in the above objects.

Another essential object of the present invention is to provide a method of treating bacterial infections due to resistant germs (for example of the S. pneumoniae type), said method consisting in the oral administration, in one or two intakes per day, of a modified-release pharmaceutical formulation, especially pediatric formulation, based on at least one active principle selected from antibiotics (for example amoxicillin optionally associated with clavulanic acid and/or at least one of its salts).

These objects are achieved by the invention, which proposes the use of modified-release microcapsules, comprising a core that contains at least one active principle AP1 formed of at least one antibiotic, and a coating for said core that governs the modified release of said active principle, for the manufacture of a drinkable or orally dispersible antibiotic pharmaceutical formulation that makes it possible to limit the increase in the antibiotic resistance of the target germs.

It is thus to the Applicant's credit to have found a novel therapeutic use for a microcapsular system for the modified release of antibiotics that is capable of extending the protection period T_{mic} beyond the T*_{mic} of an immediate-release form (IRF*) and of being formulated in the form of liquids (e.g. syrups) or suspensions to be taken orally, or in orally dispersible form. These forms are very advantageous in that they can easily be swallowed by children, the elderly or patients with a physical impediment.

This formulation can advantageously be administered in one or two (preferably two) intakes per day. Nevertheless, the formulation can also be designed to be administered in at least three intakes per day. In fact, irrespective of the number of intakes, it is desirable to increase the T_{mic} in the manner afforded by the therapeutic indication according to the invention.

Preferably, this formulation can be defined as follows, relative to an immediate-release oral formulation (IRF*) comprising at least one active principle API, and for the same dose D of AP1 as IRF*:
T_{mic}>T*_{mic}of IRF*
preferably
T_{mic}=1.1.T*_{mic}of IRF*
and particularly preferably
3.T*_{mic}of IRF*≧T_{mic}≧1.1.T*_{mic}of IRF*
where T_{mic} is the time for which the plasma concentration is greater than or equal to the minimum inhibitory concentration for a given antibiotic, and where T*_{mic} is the T_{mic} of an immediate-release oral formulation (IRF*).

According to another definition of the invention, the pharmaceutical formulation employed in the use, particularly as defined above, is an oral antibiotic pharmaceutical formulation:
on the one hand, comprises microcapsules comprising a core that contains at least one active principle AP1 formed of at least one antibiotic, and a coating for said core that governs the modified release of the active principle,
and on the other hand, can be defined, relative to an immediate-release oral formulation (IRF*) comprising active principle API, and for the same T_{mic} as IRF*, by a dose D of API such that (relative to a dose D* of active principle in IRF*):
   D<D*
   preferably
   D=0.9.D*
   and particularly preferably
   0.5.D*=D=0.8.D*

Apart from the fact that it constitutes a considerable medical advance by affording an effective treatment of bacterial infectious diseases by antibiotherapy, while at the same time stopping or at least limiting the increase in antibiotic resistance, the pharmaceutical formulation according to the invention opens the way to a regime of oral administration, one or several times a day, that favors compliance, particularly in the case of pediatric or geriatric patients.

In addition, the antibacterial efficacy, especially against resistant germs, e.g. of the Streptococcus pneumoniae, H influenzae and M. catarrhalis type, is substantially increased by virtue of the formulation obtained according to the invention, which also has the advantage of being relatively well tolerated and, finally, of offering a broad spectrum of activity.

One of the essential advantages of the invention is that it denies the logic of overdosing antibiotics in order to improve their efficacy. In this way, the therapeutic indication according to the invention puts a stop to the dramatic increase in antibiotic resistance without at the same time proposing drugs that are difficult to administer orally and therefore not conducive to compliance, or even impossible for children, the elderly or the very weak to swallow.

By virtue of this novel modified-release multimicrocapsular antibiotic pharmaceutical formulation, the invention proposes optimized pharmacokinetic profiles, especially as regards pediatric treatments, for example for amoxicillin optionally associated with clavulanic acid and/or at least one of its salts.

These results are all the more unexpected because the proposed solution runs counter to earlier recommendations, especially those in WO-A-00/61116 and WO-A-97/09042.

In terms of the invention, the essential active principle AP1 consists quantitatively and qualitatively of at least one antibiotic.

In the present disclosure, “modified release” denotes the release of medicinal active principle(s) (e.g. amoxicillin) by a pharmaceutical formulation, said release taking place at a rate slower than that of an “immediate-release” formulation, IRF, such as a conventional tablet or capsule for swallowing. Such a modified-release formulation can comprise e.g. an immediate-release phase and a slow-release phase. Modified-release formulations are well known in this field; cf., for example, Remington: The science and practice of pharmacy, 19th edition, Mack Publishing Co., Pennsylvania, USA.

In the present disclosure, “immediate release” denotes the release by an IRF of the major part of the amount of active principle in a relatively short time, for example 80% in one hour, preferably in thirty minutes, after oral ingestion. Examples of such IRFs include conventional tablets for swallowing, dispersible tablets, chewing tablets, single-dose sachets and capsules.

Advantageously, the formulation employed for the use according to the invention has a bioavailability, relative to IRF*, greater than or equal to 70%, preferably greater than or equal to 80% and particularly preferably of between 90 and 100%.

The formulation according to the invention makes it possible to improve the treatment, particularly pediatric treatment, of bacterial infections affecting e.g. the respiratory tract, namely community acquired pneumonia, acute exacerbations of chronic bronchitis, and acute bacterial sinusitis, among others. The majority of respiratory infections dealt with in outpatient clinics are caused by S. pneumoniae and/or by bacteria that produce β-lactamase, especially H. influenzae and M. catarrhalis. The pharmaceutical form according to the invention makes it possible to improve the treatment of these complaints.

The multimicrocapsular antibiotic form according to the invention comprises a coating that is designed specifically for at least one antibiotic active principle AP1 and so as to allow the sustained release of the active principle(s) AP1, on the one hand, and increase the residence time of the microcapsules in the intestinal window (small intestine) in which their bioabsorption takes place, on the other.

It is to the inventors' credit to have proposed, after lengthy and laborious research, a novel antibiotic pharmaceutical formulation, based on microcapsules for the modified release of an antibiotic AP1 (e.g. amoxicillin), that makes it possible to increase the T_{mic} for a given dose.

The object of the invention is therefore to improve the therapeutic efficacy of antibiotics without at the same time overdosing them and hence without contributing to the increase in the antibiotic resistance of the germs.

According to the invention, a particular feature of the microcapsules is that a plurality of identical microcapsules release the antibiotic continuously to give a monomodal plasma concentration profile.

In one preferred embodiment of the invention, the formulation comprises, in addition to the microcapsules, at least one active principle AP1 formed of at least one immediate-release antibiotic.

The immediate-release active principle AP1 can be, for example, in pure form (e.g. a powder) or in the form of granules in which said active principle is agglomerated with other active products and/or excipients.

Advantageously, the formulation comprises at least one immediate-release antibiotic AP1 in an amount less than or equal to 60% by weight, preferably less than or equal to 50% by weight and particularly preferably of between 0 and 40% by weight, based on the total amount of antibiotic in question.

The formulation can comprise microcapsules substantially all of which have the same in vitro release profile, or can comprise at least two types of microcapsule having different release profiles.

According to the invention, amoxicillin is a preferred antibiotic AP1 present in the core of all or some of the microcapsules of the formulation. In the preferred mode of carrying out the invention when applied to amoxicillin, the formulation comprises immediate-release amoxicillin in addition to the microcapsules of amoxicillin.

The amoxicillin capable of forming the essential active principle of the formulation according to the invention can be amoxicillin in any of its forms (e.g. amoxicillin trihydrate and/or at least one of its alkali metal salts, such as amoxicillin potassium or sodium, the latter—particularly in crystalline form—being preferred, or a mixture of the different forms of amoxicillin).

Other examples which may be mentioned of antibiotics capable of constituting the essential active principle AP1 of the formulation according to the invention are selected from the following group:

aminosalicylic acid, nalidixic acid, amoxicillin, amoxicillin and potassium clavulanate, ampicillin, ampicillin and sulbactam, azithromycin, bacampicillin, carbenicillin indanyl sodium (and other carbenicillin salts), capreomycin, cefadroxil, cefazolin, cephalexin, cephalothin, cephapirin, cephaclor, cefprozil, cephadrine, cefamandole, cefonicid, ceforanide, cefuroxime, cefixime, cefoperazone, cefotaxime, cefpodoxime, ceftazidime, ceftibuten, ceftizoxime, ceftriaxone, cefepime, cefmetazole, cefotetan, cefoxitin, ciprofloxacin, clarithromycin, clindamycin, clofazimine, cloxacillin, co-trimoxazole, cycloserine, dicloxacillin, dirithromycin, erythromycin (and erythromycin salts such as estolate, ethylsuccinate, gluceptate, lactobionate, stearate), ethambutol-HCl and other salts, ethionamide, fosfomycin, imipenem, isoniazid, levofloxacin, lomefloxacin, loracarbef, methicillin, methenamine, metronidazole, mezlocillin, nafcillin, nitrofurantoin, norfloxacin, novobiocin, ofloxacin, oxacillin, penicillin V, penicillin salts, penicillin complexes, pentamidine, piperacillin, piperacillin and tazobactam, sparfloxacin, sulfacytine, sulfamerazine, sulfamethazine, sulfamethizole, sulfasalazine, sulfisoxazole, sulfapyrazine, sulfadiazine, sulfinethoxazole, sulfapyridine, ticarcillin, ticarcillin and potassium clavulanate, trimethoprim, trimetrexate, troleandomycin, vancomycin and mixtures thereof.

In one form of implementing the use recommended according to the invention, the formulation comprises:
modified-release microcapsules whose core contains an active principle AP1 formed of an antibiotic (e.g. amoxicillin or any other antibiotic mentioned above),
and modified-release microcapsules whose core contains an active principle AP2 formed of an antibiotic (e.g. amoxicillin or any other antibiotic mentioned above) or of an active principle other than an antibiotic.

In another form of implementing the use recommended according to the invention, the formulation comprises:
modified-release microcapsules whose core contains an active principle AP1 formed of an antibiotic (e.g. amoxicillin or any other antibiotic mentioned above),
and an active principle AP2 formed of an antibiotic (e.g. amoxicillin or any other antibiotic mentioned above) or of an active principle other than an antibiotic, said AP2 being of the immediate-release type.

In yet another form of implementing the use recommended according to the invention, the formulation comprises, in addition to the essential active principle AP1, at least one other active principle AP2 preferably formed of clavulanic acid and/or at least one of its salts (preferably potassium or sodium clavularate) in the case where the essential active principle is amoxicillin.

Even more preferably, the amoxicillin:clavulanate ratio is between 2:1 and 20:1, preferably between 8:1 and 20:1 and particularly preferably between 14:1 and 16:1.

The clavulanic acid and/or at least one of its alkali metal salts, e.g. sodium or potassium clavulanate, is advantageously in crystalline form.

The association of this secondary active principle AP2 with the amoxicillin AP1 optimizes the antibiotic efficacy of the formulation. The fact that the amoxicillin is at least partly in the form of microcapsules does not detract from the bioavailability of said secondary active principle.

This non-limiting example of the association of AP1=amoxicillin and/or at least one of its salts with AP2=clavulanic acid and/or at least one of its salts in no way detracts from the fact that the invention covers any association of one or more active principles (AP), at least one of which is an antibiotic.

Likewise, if at least one antibiotic active principle is in modified-release multimicrocapsular form, any combination of one or more forms of modified-release AAP (e.g. multimicrocapsular) and/or one or more immediate-release forms of AP (e.g. antibiotic) can also be envisaged within the framework of the present invention.

According to one preferred feature of the invention that allows the formulation to be fully effective, the microcapsules of antibiotic(s) AP1—for example amoxicillin—have a particle size of between 50 nm and 800 μm, preferably of between 150 μm and 800 μm and particularly preferably of between 200 μm and 600 μm.

In terms of the invention, “particle size” is understood as meaning that a proportion of at least 75% by weight of microcapsules has a diameter between the mesh size limits in question.

Still for the purpose of improving efficacy, the amount of microcapsule coating agents advantageously represents from 1 to 50%, preferably from 5 to 40%, of the weight of the coated microcapsules. This advantageous feature is all the more difficult to obtain because the microcapsules have a large specific surface area due to their small size, accelerating the release of the microencapsulated active principle(s) AP1, for example amoxicillin.

For example, the microcapsule coating composition corresponds to one of the following three families of compositions A, B and C:

Family A:
1A—at least one film-forming polymer (P1) insoluble in the tract fluids, present in an amount of 50 to 90% and preferably 50 to 80% by dry weight, based on the total weight of the coating composition, and consisting of at least one water-insoluble cellulose derivative;
2A—at least one nitrogen-containing polymer (P2) present in an amount of 2 to 25 and preferably 5 to 15% by dry weight, based on the total weight of the coating composition, and consisting of at least one polyacrylamide and/or poly-N-vinylamide and/or poly-N-vinyllactam;
3A—at least one plasticizer present in an amount of 2 to 20 and preferably 4 to 15% by dry weight, based on the total weight of the coating composition, and consisting of at least one of the following compounds: glycerol esters, phthalates, citrates, sebacates, cetyl alcohol esters and castor oil;
4A—at least one surfactant and/or lubricant present in an amount of 2 to 20 and preferably 4 to 15% by dry weight, based on the total weight of the coating composition, and selected from anionic surfactants and/or non-ionic surfactants and/or lubricants, it being possible for said surfactant or lubricant to comprise only one of the above-mentioned products or a mixture thereof,

Family B:
1B—at least one hydrophilic polymer carrying groups ionized at neutral pH;
2B—at least one hydrophobic compound different from A;

Family C:
1C—at least one film-forming polymer insoluble in the gastrointestinal tract fluids;
2C—at least one water-soluble polymer;
3C—at least one plasticizer;
4C—optionally at least one surfactantliubricant preferably selected from the following group of products:
   anionic surfactants,
   and/or non-ionic surfactants.

For further detailed qualitative and quantitative data concerning this coating composition, reference may be made in particular to European patent EP-B-0 709 087, the content of which is integrated into the present disclosure by reference.

The coating can comprise various other additional adjuvants conventionally used in the field of coating, possible examples being pigments or fillers.

According to one advantageous modality of the invention, the microcapsule coating consists of a single layer.

The antibiotic(s) AP1, e.g. amoxicillin, used to prepare the microcapsules according to the invention can consist of pure antibiotic(s) and/or granules of antibiotic(s) prepared in a previous granulation step. Said granulation refers to conventional wet granulation processes, cf. EP-A-28 1 200 to Gist Brocades Nev., to dry compaction processes, for example using rollers, to processes involving deposition on a neutral carrier, or else to extrusion processes.

It should be noted that these granules of AP1, e.g. amoxicillin, can constitute the immediate-release non-microencapsulated part present in the formulation according to the invention.

The latter can also comprise pharmaceutically acceptable ingredients such as anticaking agents, for example talcum, colloidal silica, magnesium stearate or mixtures thereof. The amounts used can be e.g. 0.5 to 5% by weight, preferably 1.5 to 3% by weight.

The preparation of the microcapsules of antibiotic(s) AP1, e.g. amoxicillin, is amply described in the Examples below.

As far as the presentation of the formulation according to the invention is concerned, this can be powders, tablets, granules, gelatin capsules, syrups or aqueous suspensions or solutions.

The tablets can be chewing tablets and/or effervescent tablets and/or rapidly disintegrating tablets.

For administration once or twice a day in pediatrics, it is preferable to use a formulation in the form of a suspension. In this case the pharmaceutical formulation can be sold in the form of a powder to be reconstituted into a suspension by mixing with water before administration, or it can be presented directly in the form of an aqueous suspension comprising the appropriate additives, namely e.g. additives selected from surfactants, colorants, dispersants, preservatives, taste improvers, flavorings, sweeteners, antioxidants, flow promoters, texturizing agents and mixtures thereof.

According to one preferred feature of the invention:
the formulation comprises:
   microcapsules of amoxicillin,
   optionally immediate-release amoxicillin,
   immediate-release potassium clavulanate,
   and optionally excipients,
and the formulation is designed for administration twice a day.

In one preferred galenical presentation, the formulation used in the indication according to the invention takes the form of a powder (for example a single-dose sachet), a suspension or syrup, an orally dispersible tablet, a tablet dispersible in a liquid, or an effervescent tablet. In these forms or presentations given by way of example, the microcapsules can be associated with pharmaceutically acceptable excipients or carriers.

According to another of its features, the invention relates to novel pharmaceutical formulations, as defined above, for the treatment of bacterial infections in humans or animals, particularly children.

According to yet another of its features, the present invention relates to a method of treating bacterial infections in humans or animals, particularly children, said method making it possible concomitantly to limit or even stop the increase in the antibiotic resistance of the target germs.

Said method comprises the oral administration, to a subject, of a therapeutically effective amount of antibiotic(s) (for example amoxicillin) that is at least partly in the form of microcapsules eacn consisting of a core that contains at least one antibiotic AP1 (for example amoxicillin), and of a coating for said core that governs the modified release of the antibiotic AP1 (for example amoxicillin). For this purpose the formulation is in a drinkable or orally dispersible form.

Preferably, the multimicrocapsular formulation administered orally in this way can be defined as follows, relative to an immediate-release oral formulation (IRF*) comprising at least one active principle AP1 formed of at least one antibiotic, and for the same dose D of API as IRF*:
T_{mic}>T*_{mic}of IRF*
preferably
T_{mic}>1.1.T*_{mic}of IRF*
and particularly preferably
3.T*_{mic}of IRF*≧T_{mic}=1.1.T*_{mic}of IRF*

The method according to the invention for the treatment of bacterial infections, especially pediatric infections, in which the increase in antibiotic resistance is limited or even stopped, can also consist essentially in the oral administration of an oral antibiotic pharmaceutical formulation, especially pediatric formulation, for oral administration, which:
on the one hand comprises microcapsules comprising a core that contains at least one active principle AP1 formed of at least one antibiotic, and a coating for said core that governs the modified release of AP1,
and on the other hand can be defined, relative to an immediate-release oral formulation (IRF*) comprising API, and for the same T_{mic} as IRF*, by a dose D of active principle AP1 such that (relative to a dose D* of active principle AP1 in IRF*):
   D<D*
   preferably
   D=0.9.D*
   and particularly preferably
   0.5.D*=D=0.8.D*

Preferably, the formulation administered by the methods of treatment defined above comprises immediate-release AP1 (e.g. amoxicillin).

The bacterial infections in question are e.g. those affecting the respiratory tract and particularly those implicating resistant germs of the & pneumoniae, H. influenzae and M. catarrhalis type. These infections are defined above.

This formulation can advantageously be administered in one or two (preferably two) intakes per day. Nevertheless, the formulation can also be designed for administration in at least three intakes per day. In fact, irrespective of the number of intakes, it can be valuable to increase the T_{mic}, as allowed by the therapeutic indication according to the invention.

The invention will be understood more clearly with the aid of the Examples below.

### EXAMPLES

### Preparation of the Formulations According to the Invention Based on Sustained-Release Microcapsules of Amoxicillin

### Example 1

### Preparation of Formulation 1

Step 1: Granules

640 g of amoxicillin and 160 g of povidone (Plasdone K29/32) are dispersed in a water/isopropanol mixture (30/70% w/w). This solution is then sprayed onto 200 g of cellulose spheres in a Glatt GPCG1 fluidized air bed apparatus.

Step 2: Coating

930 g of granules obtained above are coated with 53.2 g of ethyl cellulose (Ethocel 7 Premium), 7.3 g of castor oil, 2.8 g of PEG 40—hydrogenated castor oil (Cremophor RH40) and 7.3 g of povidone (Plasdone K29/32), dissolved in an acetone/isopropanol mixture (60/40% w/w), in a Glatt GPCG1 fluidized air bed apparatus.

### Example 2

### Preparation of Formulation 2

Step 1: Granules

640 g of amoxicillin and 160 g of hydroxypropyl cellulose (Klucel EF) are dispersed in isopropanol. This solution is then sprayed onto 300 g of sugar spheres in a Glatt GPCG1 fluidized air bed apparatus.

Step 2: Coating

50 g of granules obtained above are coated with 2.88 g of ethyl cellulose (Ethocel 7 Premium), 0.32 g of castor oil, 1.24 g of poloxamer 188 (Lutrol F-68) and 1.92 g of povidone (Plasdone K29/32), dissolved in a water/ethanol mixture (20/80% w/w), in a miniGlatt fluidized air bed apparatus.

### Example 3

### Preparation of Formulation 3

Step 1. Granules

35 g of amoxicillin, 2.5 g of PEG 40—hydrogenated castor oil (Cremophor RH 40), 12.5 g of povidone (Plasdone K29/32) and 200 g of lactose are first mixed dry in a laboratory granulator (Mi-PRO/Pro-C-ept) for 5 minutes. This pulverulent mixture is then granulated with water (20 g). The granules are dried at 40° C. in a ventilated oven and then sized on a 500 μm mesh. The 200-500 μm fraction is selected by sieving.

Step 2: Coating

50 g of granules obtained above are coated with 1.88 g of ethyl cellulose (Ethocel 7 Premium), 0.23 g of castor oil, 0.75 g of PEG 40—hydrogenated castor oil (Cremophor RH 40) and 0.90 g of povidone (Plasdone K29/32), dissolved in an acetone/isopropanol mixture (60/40% w/w), in a miniGlatt fluidized air bed apparatus.

### Example 4

### Preparation of Formulation 4

Step 1. Granules

640 g of amoxicillin and 160 g of povidone (Plasdone K29/32) are dispersed in a water/isopropanol mixture (30/70% w/w). This solution is then sprayed onto 200 g of cellulose spheres in a Glatt GPCG1 fluidized air bed apparatus.

Step 2: Coating

850 g of granules obtained above are coated with 384.9 g of ethyl cellulose in aqueous dispersion (Aquacoat ECD 30, i.e. 117 g of dry extract), 28.5 g of dibutyl sebacate and 4.5 g of povidone (Plasdone K29/32) in a Glatt GPCG1 fluidized air bed apparatus.

### Example 5

### Preparation of Formulation 5

Step 1: Granules

700 g of amoxicillin, 300 g povidone (Plasdone K29/32) and 200 g of water are mixed in a laboratory mixer (Kitchen-Aid) for 5 minutes. This pasty mixture is extruded through a 0.5 mm mesh by means of a 20 extruder (Caleva). The filaments obtained are then spheronized by means of a 250 spheronizer (Caleva). The particles obtained are dried at 40° C. in a fluidized air bed. The 300-700 μm fraction is selected by sieving.

Step 2: Coating

450 g of granules obtained above are coated with 35 g of ethyl cellulose (Ethocel 20 Premium), 5 g of dibutyl sebacate and 10 g of PEG 35000, dissolved in a water/ethanol mixture (20/80% w/w), in an Aeromatic-Fielder MP1 fluidized air bed apparatus.

### Example 6

### Preparation of Formulation 6

Step 1: Granules

590 g of amoxicillin and 10 g of magnesium stearate are mixed in a laboratory mixer (Kitchen-Aid) for 5 minutes. This mixture is then compacted by means of an Alexenderwerk WP120 laboratory compactor. The product obtained is then granulated by means of an Erweka oscillating granulator equipped with a 500 μm mesh. The 100-500 μm fraction of the product obtained is selected by sieving.

Step 2: Coating

450 g of granules obtained above are coated with 35 g of ethyl cellulose (Ethocel 20 Premium), 5 g of dibutyl sebacate and 10 g of PEG 35000, dissolved in a water/ethanol mixture (20/80% w/w), in an Aeromatic-Fielder MP1 fluidized air bed apparatus.

### Example 7

### Preparation of Formulation 7

Step 1: Granules

590 g of amoxicillin and 10 g of magnesium stearate are mixed in a laboratory mixer (Kitchen-Aid) for 5 minutes. This mixture is compacted by means of an Alexenderwerk WP120 laboratory compactor. The product obtained is then granulated by means of an Erweka oscillating granulator equipped with a 500 μm mesh. The 100-500 μm fraction of the product obtained is selected by sieving.

Step 2: Coating

450 g of granules obtained above are coated with 60 g of Eudragit RS100, 4 g of triethyl citrate and 16 g of PEG 35000, dissolved in isopropanol, in an Aeromatic-Fielder MP1 fluidized air bed apparatus.

### Example 8

### Preparation of Formulation 8 Comprising an Amoxicillin Fraction in the Form of Modified-Release (Sustained-Release) Microcapsules and an Amoxicillin Fraction in the Form of Immediate-Release Amoxicillin

300 g of granules prepared in step 1 of Example 4 for the manufacture of micro-capsules are mixed with 700 g of microencapsulated amoxicillin corresponding to Formulation 4 of Example 4.

### Example 9

### Plasma Profiles of the Multimicrocapsular Amoxicillin Formulations According to the Invention

The plasma amoxicillin concentration profile after the administration of modified-release microcapsules according to the invention was simulated from the following data:

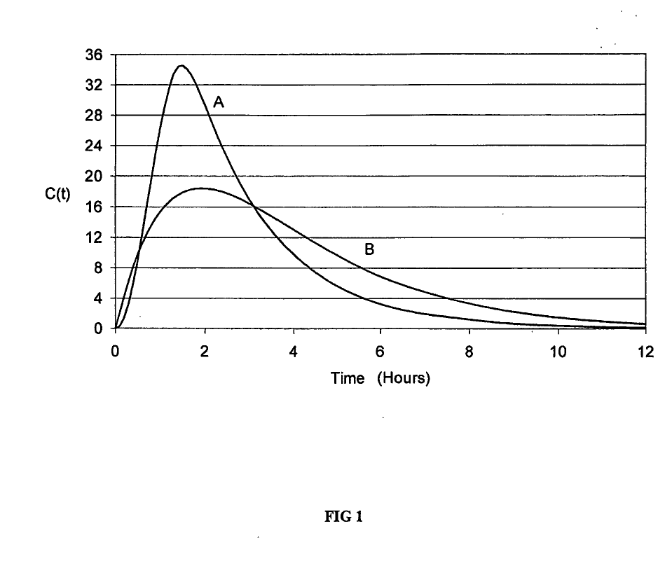

The dose administered is 43 mg/kg. The modified-release microcapsules release 39% of the dose in one hour and 50% of the dose in 1.4 hours. The plasma profile resulting from an IV injection (“response function IV”) is a monoexponential function with an elimination half-life of 1.3 hours. Finally, the plasma profile is calculated by convolution of the amoxicillin release rate by the IV response function IV. The plasma profile obtained after administration of the modified-release microcapsules is compared in FIG. 1 with the profile resulting from the administration of the same dose of an immediate-release oral form IRF*. It is seen that the modified-release form according to the invention affords an increase in the time T_{mic}. For example, for MIC=4 μg/ml, T_{mic} is 5.3 hours for the immediate-release form and 7.3 hours for the form according to the invention. The modified-release microcapsular amoxicillin formulations according to the invention are therefore more effective, for the same dose, than the immediate-release amoxicillin formulations IRF*.

FIG. 1 attached shows two plasma amoxicillin concentration profiles A and B (μg/ml) resulting from the administration of 43 mg/kg, profile A being that of an immediate-release oral form (IRF*) and profile B being that of a modified-release microcapsular oral form of the type whose use is proposed according to the invention for the manufacture of an effective antibiotic drug.

1. Use of modified-release microcapsules, comprising a core that contains at least one active principle AP1 formed of at least one antibiotic, and a coating for said core that governs the modified release of said active principle, for the manufacture of a drinkable or orally dispersible antibiotic pharmaceutical formulation that makes it possible to limit the increase in the antibiotic resistance of the target germs.

2. Use according to claim 1, characterized in that this formulation can be defined as follows, relative to an immediate-release oral formulation (IRF*) comprising at least one active principle AP1, and for the same dose D of AP1 as IRF*:
T_{mic}>T*_{mic}of IRF*
preferably
T_{mic}>1.1.T*_{mic}of IRF*
and particularly preferably
3.T*_{mic}of IRF*>T_{mic}>1.1.T*_{mic}of IRF*
where T_{mic} is the time for which the plasma concentration is greater than or equal to the minimum inhibitory concentration for a given antibiotic, and where T*_{mic} is the T_{mic} of an immediate-release oral formulation (IRF*).

3. Use according to claim 1, characterized in that the microcapsules have a particle size of between 50 nm and 800 μm.

4. Use according to claim 1, characterized in that, in addition to the microcapsules, the formulation comprises at least one active principle AP1 formed of at least one immediate-release antibiotic.

5. Use according to claim 4, characterized in that the formulation comprises at least one immediate-release antibiotic AP1 in an amount less than or equal to 60% by weight, preferably less than or equal to 50% by weight and particularly preferably of between 0 and 40% by weight, based on the total amount of antibiotic AP1.

6. Use according to claim 1, characterized in that the core of the microcapsules comprises, as active principle AP1, an antibiotic selected from the following group:
aminosalicylic acid, nalidixic acid, amoxicillin, amoxicillin and potassium clavulanate, ampicillin, ampicillin and sulbactam, azithromycin, bacampicillin, carbenicillin indanyl sodium (and other carbenicillin salts), capreomycin, cefadroxil, cefazolin, cephalexin, cephalothin, cephapirin, cephaclor, cefprozil, cephadrine, cefamandole, cefonicid, ceforanide, cefuroxime, cefixime, cefoperazone, cefotaxime, cefpodoxime, ceftazidime, ceftibuten, ceftizoxime, ceftriaxone, cefepime, cefmetazole, cefotetan, cefoxitin, ciprofloxacin, clarithromycin, clindamycin, clofazimine, cloxacillin, co-trimoxazole, cycloserine, dicloxacillin, dirithromycin, erythromycin (and erythromycin salts such as estolate, ethylsuccinate, gluceptate, lactobionate, stearate), ethambutol-HCl and other salts, ethionamide, fosfomycin, imipenem, isoniazid, levofloxacin, lomefloxacin, loracarbef, methicillin, methenamine, metronidazole, mezlocillin, nafcillin, nitrofurantoin, norfloxacin, novobiocin, ofloxacin, oxacillin, penicillin V, penicillin salts, penicillin complexes, pentamidine, piperacillin, piperacillin and tazobactam, sparfloxacin, sulfacytine, sulfamerazine, sulfamethazine, sulfamethizole, sulfasalazine, sulfisoxazole, sulfapyrazine, sulfadiazine, sulfmethoxazole, sulfapyridine, ticarcillin, ticarcillin and potassium clavulanate, trimethoprim, trimetrexate, troleandomycin, vancomycin and mixtures thereof.

7. Use according to claim 1, characterized in that the formulation comprises:
modified-release microcapsules whose core contains an active principle AP1 formed of an antibiotic,
and modified-release microcapsules whose core contains an active principle AP2 formed of an antibiotic or of an active principle other than an antibiotic.

8. Use according to claim 1, characterized in that the formulation comprises:
modified-release microcapsules whose core contains an active principle AP1 formed of an antibiotic,
and an active principle AP2 formed of an antibiotic or of an active principle other than an antibiotic, said AP2 being of the immediate-release type.

9. Use according to claim 1, characterized in that the core of the microcapsules comprises amoxicillin as active principle AP1.

10. Use according to claim 1, characterized in that the formulation comprises at least one other antibiotic AP2 preferably formed of clavulanic acid and/or at least one of its salts (preferably potassium clavulanate), and even more preferably in an arnoxicillin:clavulanate ratio of between 2:1 and 20:1, preferably of between 8:1 and 20:1 and particularly preferably of between 14:1 and 16:1.

11. Use according to claim 1, characterized in that the formulation is in the form of powders, tablets, granules, gelatin capsules, syrups or aqueous suspensions.

12. Use according to claim 10, characterized in that:
the formulation comprises:
micro capsules of amoxicillin,
optionally immediate-release amoxicillin,
immediate-release potassium clavulanate,
and optionally excipients,
and the formulation is designed for administration twice a day.

13. Use according to claim 1, characterized in that the microcapsule coating consists of a single layer.
